(19) United States
(12) Patent Application Publication
(10) Pub. No.: US 2022/0249440 A1
(43) Pub. Date: Aug. 11, 2022

(54) MUSCLE REGENERATION PROMOTER

(71) Applicants: SATO PHARMACEUTICAL CO., LTD., Tokyo (JP); KEIO UNIVERSITY, Tokyo (JP)

(72) Inventors: Kazumasa OKUBO, Shinagawa-ku, Tokyo (JP); Kazuki YUASA, Shinagawa-ku, Tokyo (JP); Yoshiki ITOH, Shinagawa-ku, Tokyo (JP); Masaya NAKAMURA, Shinjuku-ku, Tokyo (JP); Keisuke HORIUCHI, Shinjuku-ku, Tokyo (JP)

(73) Assignees: SATO PHARMACEUTICAL CO., LTD., Tokyo (JP); KEIO UNIVERSITY, Tokyo (JP)

(21) Appl. No.: 17/623,597

(22) PCT Filed: Jun. 25, 2020
(86) PCT No.: PCT/JP2020/025003

§ 371 Date: Dec. 28, 2021

(30) Foreign Application Priority Data
Jun. 28, 2019 (JP) 2019-121327

(51) Int. Cl. A61K 31/4045; A61P 21/00; A61K 31/55

(52) U.S. Cl. CPC A61K 31/4045; A61K 31/55; A61P 21/00

(57) ABSTRACT

The present invention is to provide a means for promoting muscle regeneration from muscle damage.

According to the present invention, the muscle regeneration from muscle damage is promoted by using a compound having 5-HT_{2B} receptor agonist activity or a salt thereof is used as an active ingredient.

### TECHNICAL FIELD

The present invention relates to a muscle regeneration promoter comprising a compound having 5-HT_{2B} receptor agonist activity or a pharmaceutically acceptable salt thereof as an active ingredient.

### BACKGROUND ART

Muscle damage is clinically classified into those caused by muscle strain (pulled muscle), high-energy injury, surgical operation, and the like. Muscle damage is known to cause various complications (such as dysfunction, muscular atrophy, and local pain) (Non Patent Literature 1).

In such muscle damages, particularly, the muscle strain caused by blunt external force (bruise) is sports injury caused most frequently (Non Patent Literature 2). Although there are no accurate statistics, it is considered that the number of the patients in Japan is around tens of thousands per year.

As the first aid for muscle damage, RICE treatment (Rest, Icing, Compression, and Elevation) has been recommended so far. After the first aid, symptomatic treatment for pain, rehabilitation, and the like have been conducted.

Muscle tissue has a mechanism for regeneration from the damage. It is known that muscle satellite cells are essential for muscle regeneration. The muscle satellite cells that reside in the vicinity of the muscle-fiber basement membrane, exist in a quiescent state under normal conditions, but when muscle damage is caused, the cells are activated and differentiated into myoblasts, and form muscle fibers via cell fusion. When the muscle regeneration is completed, the remaining muscle satellite cells enter the quiescent state again (Non Patent Literatures 3 and 4).

As the factor that promotes muscle regeneration, a hepatocyte growth factor (HGF) (Non Patent Literature 5), and an insulin-like growth factor 1 (IGF-1) (Non Patent Literature 6) are known. Further, it has been reported that muscle hypertrophy can be induced by suppressing a factor that inhibits muscle regeneration (Patent Literature 1).

However, it takes a long time to regenerate the muscle by the above mechanism. As a result, conventional treatments that do not take particular measures to promote muscle regeneration cause muscle weakness and delay the return of muscle damaged patients to daily life and sports activities.

Further, if it takes time to regenerate the muscle, part of the muscle tissue may be replaced with the scar tissue derived from collagen that has remained in the muscle tissue for a long period of time. Since the scar tissue reduces the strength of plastic muscles, the risk of recurrence of muscle damage is high (Non Patent Literature 1).

### CITATION LIST

### Patent Literature

Patent Literature 1: WO 2013/039244 A

### Non Patent Literature

Non Patent Literature 1: J. Appl. Physiol., Vol. 95, No. 2, pp. 771-780, 2003.
Non Patent Literature 2: Am. J. Sports Med., Vol. 27, No. 1, pp. 2-9, 1999.
Non Patent Literature 3: Am. J. Sports Med., Vol. 33, No. 5, pp. 745-64, May 2005.
Non Patent Literature 4: J. Bone Joint Surg. Am., Vol. 84-A, No. 5, pp. 822-32, May 2002.
Non Patent Literature 5: Dev. Biol., Vol. 194, No. 1, pp. 114-128, 1998.
Non Patent Literature 6: J. Cell. Physiol., Vol. 138, No. 2, pp. 311-5, February 1989.

### SUMMARY OF THE INVENTION

### Technical Problem

The conventional treatments that takes time to regenerate the muscle cause muscle weakness easily, decrease motor function of patients and shorten healthy life expectancy, or cause the long-term suspension of activities of athletes easily. Thus a more effective novel treatment has been desired. Further, although the molecular mechanism for muscle regeneration has been widely studied, a drug effective in the muscle regeneration has not been developed yet. Therefore, the development of a drug that promotes muscle regeneration has been strongly desired.

### Solution to Problem

The present inventors performed intensive studies to solve the problems, and as a result, found that muscle regeneration is promoted when a compound having 5-HT_{2B} receptor agonist activity or a salt thereof is used, and completed the present invention. That is, the present invention relates to the following [1] to [11].

[1] A muscle regeneration promoter, comprising a compound having 5-HT_{2B} receptor agonist activity, or a pharmaceutically acceptable salt thereof.

[2] The muscle regeneration promoter described in the above [1], wherein the compound having 5-HT_{2B} receptor agonist activity is a compound represented by the general formula (I):

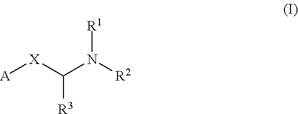

[wherein

R¹ represents a hydrogen atom, a lower alkyl group, or a group represented by the general formula: —CH₂Ar (wherein Ar represents a phenyl group that may be substituted with a hydroxyl group);

R² represents a hydrogen atom, or a lower alkyl group;

R³ represents a hydrogen atom, or a lower alkyl group;

X represents a methylene group, a group represented by the general formula: —C(R⁴)═CH—, a group represented by the general formula: —N(R⁴)—CH₂—, a group represented by the general formula: —O—CH(R⁴)—, or a group represented by the general formula: —CH(R⁴)—;

R⁴ represents a hydrogen atom or a phenyl group, or R⁴ and R¹ may be together to form a group represented by the general formula: —(CH₂)ₙ— (wherein n is an integer of 1 or 2); and

A is a group represented by the general formula (II):

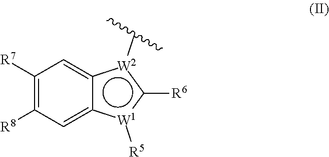

(wherein

W¹ and W² represent each independently a nitrogen atom, or a carbon atom (wherein when one of W¹ and W² represents a nitrogen atom, the remaining represents a carbon atom);

R⁵ represents a hydrogen atom, or a lower alkyl group;

R⁶ represents a hydrogen atom or a lower alkyl group, or R⁶ and R¹ may be together to form a group represented by the general formula: —(CH₂)ₚ— (wherein p is an integer of 1 or 2);

R⁷ represents a hydrogen atom, a halogen atom, a hydroxyl group, a lower alkoxy group, a carbamoyl group, or a group represented by the general formula: —Y—(CH₂)_{q}—R⁹ (wherein Y represents a single bond, an oxygen atom, a sulfur atom, or a group represented by the general formula: —N(R^{N})— (wherein R^{N} represents a hydrogen atom or a lower alkyl group), and q is an integer of 1 or 2);

R⁸ represents a hydrogen atom, or a halogen atom; and

R⁹ represents a phenyl group, a thienyl group, or a furyl group); or

a group represented by the general formula (III):

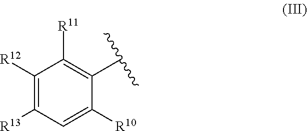

(wherein

R¹⁰ represents a hydrogen atom or a lower alkoxy group, or R¹⁰ and R¹ or R³ may be together to form a group represented by the general formula: —(CH₂)ᵣ— (wherein r is an integer of 1 or 2);

R¹¹ represents a hydrogen atom or a hydroxyl group, or R¹¹ and R¹² may be together to form a propylene group;

R¹² represents a hydrogen atom, a halogen atom, a hydroxyl group, a lower alkoxy group, or a halo-lower alkyl group; and

R¹³ represents a hydrogen atom, a halogen atom, or a cyano group).]

[3] The muscle regeneration promoter described in the above [2], wherein A is a group represented by the general formula (II).

[4] The muscle regeneration promoter described in the above [1], wherein the compound having 5-HT_{2B} receptor agonist activity is selected from the group consisting of the following compounds <1> to <21>:

<1> 3-(2-aminoethyl)-1H-indol-5-ol,

<2> 3-(2-aminopropyl)-1H-indol-5-ol,

<3> 2-(1H-indol-3-yl)ethan-1-amine,

<4> 3-(2-aminoethyl)-1H-indol-5-carboxamide,

<5> 3-(2-aminoethyl)-2-methyl-1H-indol-5-ol,

<6> 1-(5-(thiophen-2-ylmethoxy)-1H-indol-3-yl)propan-2-amine,

<7> 5-methoxy-3-(1,2,3,6-tetrahydropyridin-4-yl)-1H-indole,

<8> (S)-1-(6-chloro-5-fluoro-1H-indol-1-yl)propan-2-amine,

<9> (S)-1-(5,6-difluoro-1H-indol-1-yl)propan-2-amine,

<10> 6-methyl-1,2,3,4,5,6-hexahydroazepino[4,5-b]indole,

<11> 1-(3-chlorophenyl)piperazine,

<12> 1-(3-(trifluoromethyl)phenyl)piperazine,

<13> 1-(4-iodo-2,5-dimethoxyphenyl)propan-2-amine,

<14> 1-(4-bromo-2,5-dimethoxyphenyl)propan-2-amine,

<15> (R)-8-chloro-3-methyl-5-phenyl-2,3,4,5-tetrahydro-1H-benzo[d]azepin-7-ol,

<16> (R)-7-(dipropylamino)-5,6,7,8-tetrahydronaphthalen-1-ol,

<17> (S)-1-(3-(trifluoromethyl)phenyl)propan-2-amine,

<18> (S)-3-((5-methoxy-2,3-dihydro-1 H-inden-4-yl)oxy)pyrrolidine,

<19> 2-chloro-6-(piperazin-1-yl)pyrazine,

<20> 1-(6-chloro-5-(trifluoromethyl)pyridin-2-yl)piperazine, and

<21> 2-(piperazin-1-yl)quinoline.

[5] The muscle regeneration promoter described in any one of the above [1] to [4], wherein the compound having 5-HT_{2B} receptor agonist activity is selected from the group consisting of the following compounds <a> to <d>:

<a> 3-(2-aminopropyl)-1H-indol-5-ol,

<b> 1-(5-(thiophen-2-ylmethoxy)-1H-indol-3-yl)propan-2-amine,

<c> (S)-1-(6-chloro-5-fluoro-1H-indol-1-yl)propan-2-amine, and

<d> 6-methyl-1,2,3,4,5,6-hexahydroazepino[4,5-b]indole.

[6] The muscle regeneration promoter described in any one of the above [1] to [5], wherein muscle regeneration after muscle damage or in myogenic disease is promoted.

[7] The muscle regeneration promoter described in the above [6], wherein the muscle regeneration after muscle damage is promoted.

[8] The muscle regeneration promoter described in the above [7], wherein the muscle damage is muscle strain.

[9] A therapeutic agent for muscle damage, comprising a compound having 5-HT_{2B} receptor agonist activity, or a pharmaceutically acceptable salt thereof.

[10] The therapeutic agent for muscle damage described in the above [9], wherein the compound having 5-HT_{2B} receptor agonist activity is a compound represented by the general formula (I):

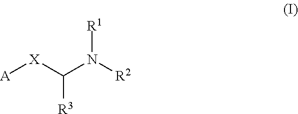

[wherein

R¹ represents a hydrogen atom, a lower alkyl group, or a group represented by the general formula: —CH₂Ar (wherein Ar represents a phenyl group that may be substituted with a hydroxyl group);

R² represents a hydrogen atom, or a lower alkyl group;

R³ represents a hydrogen atom, or a lower alkyl group;

X represents a methylene group, a group represented by the general formula: —C(R⁴)═CH—, a group represented by the general formula: —N(R⁴)—CH₂—, a group represented by the general formula: —O—CH(R⁴)—, or a group represented by the general formula: —CH(R⁴)—;

R⁴ represents a hydrogen atom or a phenyl group, or R⁴ and R¹ may be together to form a group represented by the general formula: —(CH₂)ₙ— (wherein n is an integer of 1 or 2); and

A is a group represented by the general formula (II):

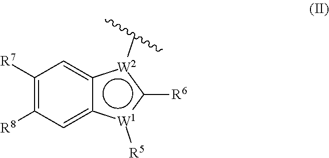

(wherein

W¹ and W² represent each independently a nitrogen atom, or a carbon atom (wherein when one of W¹ and W² represents a nitrogen atom, the remaining represents a carbon atom);

R⁵ represents a hydrogen atom, or a lower alkyl group;

R⁶ represents a hydrogen atom or a lower alkyl group, or R⁶ and R¹ may be together to form a group represented by the general formula: —(CH₂)ₚ— (wherein p is an integer of 1 or 2);

R⁷ represents a hydrogen atom, a halogen atom, a hydroxyl group, a lower alkoxy group, a carbamoyl group, or a group represented by the general formula: —Y—(CH₂)_{q}—R⁹ (wherein Y represents a single bond, an oxygen atom, a sulfur atom, or a group represented by the general formula: —N(R^{N})— (wherein R^{N} represents a hydrogen atom or a lower alkyl group), and q is an integer of 1 or 2);

R⁸ represents a hydrogen atom, or a halogen atom; and

R⁹ represents a phenyl group, a thienyl group, or a furyl group); or

a group represented by the general formula (III):

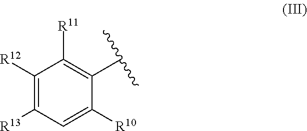

(wherein

R¹⁰ represents a hydrogen atom or a lower alkoxy group, or R¹⁰ and R¹ or R³ may be together to form a group represented by the general formula: —(CH₂)ᵣ— (wherein r is an integer of 1 or 2);

R¹¹ represents a hydrogen atom or a hydroxyl group, or R¹¹ and R¹² may be together to form a propylene group;

R¹² represents a hydrogen atom, a halogen atom, a hydroxyl group, a lower alkoxy group, or a halo-lower alkyl group; and

R¹³ represents a hydrogen atom, a halogen atom, or a cyano group).]

[11] The therapeutic agent for muscle damage described in the above [9] or [10], wherein the muscle damage is muscle strain.

### Effects of Invention

As shown in Examples to be described later, the muscle regeneration promoter according to the present invention can promote muscle regeneration.

### BRIEF DESCRIPTION OF THE DRAWINGS

FIG. 1 is a histogram of the regenerated single-muscle fiber area after administration of BW723C86.

FIG. 2 shows the mean muscle fiber area of regenerated muscle after administration of BW723C86.

FIG. 3 is a histogram of the regenerated single-muscle fiber area after administration of α-methyl-5-HT.

FIG. 4 shows the mean muscle fiber area of regenerated muscle after administration of α-methyl-5-HT.

FIG. 5 is a histogram of the regenerated single-muscle fiber area after administration of Ro 60-0175.

FIG. 6 shows the mean muscle fiber area of regenerated muscle after administration of Ro 60-0175.

FIG. 7 is a histogram of the regenerated single-muscle fiber area after administration of PNU 22394.

FIG. 8 shows the mean muscle fiber area of regenerated muscle after administration of PNU 22394.

### DESCRIPTION OF EMBODIMENTS

<Active Ingredient>

The muscle regeneration promoter according to the present invention contains a compound having 5-HT_{2B} receptor agonist activity or a pharmaceutically acceptable salt thereof as an active ingredient.

The term “5-HT_{2B} receptor” used herein is one of the receptors of 5-hydroxytryptamine (also known as serotonin).

The term “compound having 5-HT_{2B} receptor agonist activity” used herein means a compound that binds to a serotonin receptor 5-HT_{2B} and activates the receptor.

The term “compound having 5-HT_{2B} receptor agonist activity” used herein is also referred to as “5-HT_{2B} receptor agonist”.

The term “compound having 5-HT_{2B} receptor agonist activity” (hereinafter, also referred to as “5-HT_{2B} receptor agonist”) may also have agonist activity against a serotonin receptor other than the 5-HT_{2B} receptor.

The presence or absence of the “5-HT_{2B} receptor agonist activity” can be measured and determined in accordance with the test method disclosed in the literature (Br. J. Pharmacol., Vol. 128, No. 1, pp. 13-20, September 1999).

The compound having 5-HT_{2B} receptor agonist activity is preferably represented by the general formula (I).

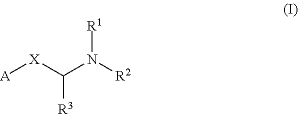

[wherein

R¹ represents a hydrogen atom, a lower alkyl group, or a group represented by the general formula: —CH₂Ar (wherein Ar represents a phenyl group that may be substituted with a hydroxyl group);

R² represents a hydrogen atom, or a lower alkyl group;

R³ represents a hydrogen atom, or a lower alkyl group;

X represents a methylene group, a group represented by the general formula: —C(R⁴)═CH—, a group represented by the general formula: —N(R⁴)—CH₂—, a group represented by the general formula: —O—CH(R⁴)—, or a group represented by the general formula: —CH(R⁴)—;

R⁴ represents a hydrogen atom or a phenyl group, or R⁴ and R¹ may be together to form a group represented by the general formula: —(CH₂)ₙ— (wherein n is an integer of 1 or 2); and

A is a group represented by the general formula (II):

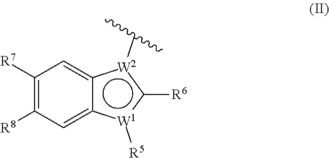

(wherein

W¹ and W² represent each independently a nitrogen atom, or a carbon atom (wherein one of W¹ and W² represents a nitrogen atom, the remaining represents a carbon atom);

R⁵ represents a hydrogen atom, or a lower alkyl group;

R⁶ represents a hydrogen atom or a lower alkyl group, or R⁶ and R¹ may be together to form a group represented by the general formula: —(CH₂)ₚ— (wherein p is an integer of 1 or 2);

R⁷ represents a hydrogen atom, a halogen atom, a hydroxyl group, a lower alkoxy group, a carbamoyl group, or a group represented by the general formula: —Y—(CH₂)_{q}—R⁹ (wherein Y represents a single bond, an oxygen atom, a sulfur atom, or a group represented by the general formula: —N(R^{N})— (wherein R^{N} represents a hydrogen atom or a lower alkyl group), and q is an integer of 1 or 2);

R⁸ represents a hydrogen atom, or a halogen atom; and

R⁹ represents a phenyl group, a thienyl group, or a furyl group); or

a group represented by the general formula (III):

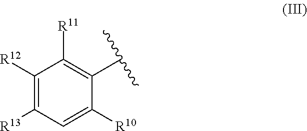

(wherein

R¹⁰ represents a hydrogen atom or a lower alkoxy group, or R¹⁰ and R¹ or R³ may be together to form a group represented by the general formula: —(CH₂)ᵣ— (wherein r is an integer of 1 or 2);

R¹¹ represents a hydrogen atom or a hydroxyl group, or R¹¹ and R¹² may be together to form a propylene group;

R¹² represents a hydrogen atom, a halogen atom, a hydroxyl group, a lower alkoxy group, or a halo-lower alkyl group; and

R¹³ represents a hydrogen atom, a halogen atom, or a cyano group).]

Hereinafter, the meanings of terms used in the above general formulas will be described below.

The term “lower alkyl group” used herein means a linear or branched alkyl group having 1 to 6 carbon atoms, and for example, a methyl group, an ethyl group, a propyl group, an isopropyl group, a butyl group, an isobutyl group, a sec-butyl group, a tert-butyl group, a pentyl group, an isopentyl group, an isoamyl group, a neopentyl group, a 1,1-dimethylpropyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,2,2-trimethylpropyl group, and a 1-ethyl-3-methylpropyl group, etc. are mentioned.

“halogen atom” used herein include a fluorine atom, a chlorine atom, a bromine atom, and an iodine atom, and the like.

The term “lower alkoxy group” used herein means a group in which the hydrogen atom of a hydroxyl group is substituted by the above-mentioned “lower alkyl group”, and for example, a methoxy group, an ethoxy group, a propoxy group, an isopropoxy group, a butoxy group, a sec-butoxy group, a tert-butoxy group, a pentyloxy group, an isopentyloxy group, a hexyloxy group, and an isohexyloxy group, etc. are mentioned.

The term “halo-lower alkyl group” used herein means the above-mentioned “lower alkyl group” in which the substitutable optional position(s) is/are substituted by 1 or 2 or more, preferably 1 to 5 of identical or different halogen atom(s) mentioned above, and for example, a fluoromethyl group, a difluoromethyl group, a trifluoromethyl group, a 2-fluoroethyl group, a 1,2-difluoroethyl group, a 2,2,2-trifluoroethyl group, a pentafluoroethyl group, a chloromethyl group, a 2-chloroethyl group, a 1,2-dichloroethyl group, a 2,2,2-trichloroethyl group, a bromomethyl group, and an iodomethyl group, etc. are mentioned.

The term “substitutable optional position(s)” used herein means site(s) of substitutable hydrogen atom(s) on a carbon atom, a nitrogen atom, an oxygen atom, and/or a sulfur atom, where the substitution of the hydrogen atom(s) is/are chemically accepted, and consequently a stable compound is brought.

To specifically disclose the “compound having 5-HT_{2B} receptor agonist activity”, the respective symbols used in the general formula (I) and the like will be described in detail with referring to their preferable specific examples thereof.

R¹ represents a hydrogen atom, a lower alkyl group, or a group represented by the general formula: —CH₂Ar.

As the lower alkyl group for R¹, for example, a methyl group, an ethyl group, a propyl group, an isopropyl group, a butyl group, an isobutyl group, a sec-butyl group, a tert-butyl group, a pentyl group, an isopentyl group, an isoamyl group, a neopentyl group, a 1,1-dimethylpropyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,2,2-trimethylpropyl group, and a 1-ethyl-3-methylpropyl group, etc. are mentioned, and a propyl group is preferable.

R¹ may be a group represented by the general formula: —CH₂Ar.

Ar represents a phenyl group that may be substituted with a hydroxyl group. That is, Ar represents an unsubstituted phenyl group or a phenyl group substituted with a hydroxyl group, and examples of the Ar include a phenyl group, a 2-hydroxyphenyl group, a 3-hydroxyphenyl group, and a 4-hydroxyphenyl group.

As the group represented by the general formula: —CH₂Ar, for example, a benzyl group, a 2-hydroxybenzyl group, a 3-hydroxybenzyl group, and a 4-hydroxybenzyl group, are mentioned, and a 2-hydroxybenzyl group is preferable.

R¹ is preferably a hydrogen atom, a propyl group, or a 2-hydroxybenzyl group.

R² represents a hydrogen atom, or a lower alkyl group.

As the lower alkyl group for R², for example, a methyl group, an ethyl group, a propyl group, an isopropyl group, a butyl group, an isobutyl group, a sec-butyl group, a tert-butyl group, a pentyl group, an isopentyl group, an isoamyl group, a neopentyl group, a 1,1-dimethylpropyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,2,2-trimethylpropyl group, and a 1-ethyl-3-methylpropyl group, etc. are mentioned, and a methyl group, and a propyl group are preferable.

R² is preferably a hydrogen atom, a methyl group, or a propyl group.

R³ represents a hydrogen atom, or a lower alkyl group.

As the lower alkyl group for R³, for example, a methyl group, an ethyl group, a propyl group, an isopropyl group, a butyl group, an isobutyl group, a sec-butyl group, a tert-butyl group, a pentyl group, an isopentyl group, an isoamyl group, a neopentyl group, a 1,1-dimethylpropyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,2,2-trimethylpropyl group, and a 1-ethyl-3-methylpropyl group, etc. are mentioned, and a methyl group is preferable.

R³ is preferably a hydrogen atom, or a methyl group.

X represents a methylene group, a group represented by the general formula: —C(R⁴)═CH—, a group represented by the general formula: —N(R⁴)—CH₂—, a group represented by the general formula: —O—CH(R⁴)—, or a group represented by the general formula: —CH(R⁴)—.

R⁴ represents a hydrogen atom or a phenyl group, or R⁴ and R¹ may be together to form a group represented by the general formula: —(CH₂)ₙ— (wherein n is an integer of 1 or 2).

In this regard, the general formula (I) is expressed on the basis of the specific kind of X.

(1) When X is a methylene group, the general formula (I) is represented by the following general formula (I-1).

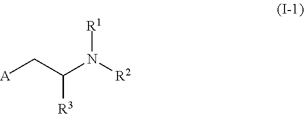

(2) When X is a group represented by the general formula: —C(R⁴)═CH—, the general formula (I) is represented by the following general formula (I-2).

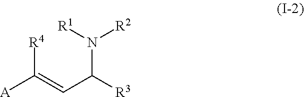

(3) When X is a group represented by the general formula: —N(R⁴)—CH₂—, the general formula (I) is represented by the following general formula (I-3).

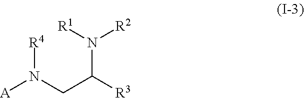

(4) When X is a group represented by the general formula: —O—CH(R⁴)—, the general formula (I) is represented by the following general formula (I-4).

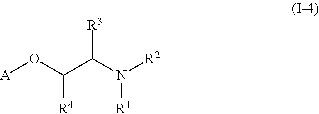

(5) When X is a group represented by the general formula: —CH(R⁴)—, the general formula (I) is represented by the following general formula (I-5).

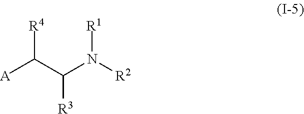

The expression “R⁴ and R¹ may be together to form a group represented by the general formula: —(CH₂)ₙ—” means that a ring is provided by the group represented by the general formula: —(CH₂)ₙ— formed of R¹ and R⁴. As the specific examples, the groups represented by the following general formulas (I-6) to (I-8) are mentioned.

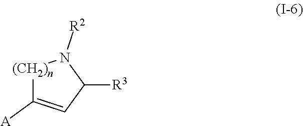

The general formula (I-6) corresponds to the general formula (I) (in the formula, X is a group represented by the general formula: —C(R⁴)═CH—, R¹ and R⁴ are together to form a group represented by the general formula: —(CH₂)ₙ—, and further, n is an integer of 1 or 2, and preferably 2).

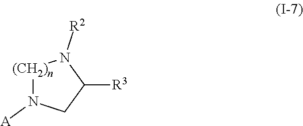

The general formula (I-7) corresponds to the general formula (I) (in the formula, X is a group represented by the general formula: —N(R⁴)—CH₂—, R¹ and R⁴ are together to form a group represented by the general formula: —(CH₂)ₙ—, and further, n is an integer of 1 or 2, and preferably 2).

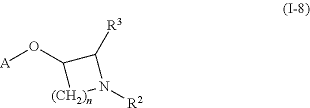

The general formula (I-8) corresponds to the general formula (I) (in the formula, X is a group represented by —O—CH(R⁴)—, R¹ and R⁴ are together to form a group represented by the general formula: —(CH₂)ₙ—, and further, n is an integer of 1 or 2, and preferably 2).

A is a group represented by the following general formula (II) or (III).

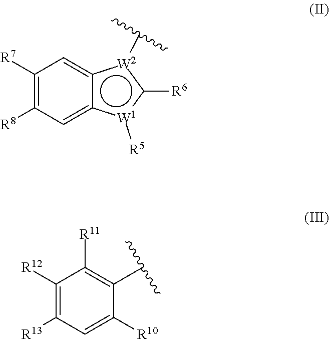

The terms used in the above general formulas (II) and (III) will be described below.

In the general formula (II), W¹ and W² each represent independently a nitrogen atom, or a carbon atom. When one of W¹ and W² represents a nitrogen atom, the remaining represents a carbon atom.

Herein, a combination of W¹ and W² will be described.

When W¹ is a nitrogen atom and W² is a carbon atom, the general formula (II) is represented by the following general formula (II-1).

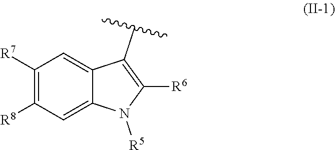

When W¹ is a carbon atom and W² is a nitrogen atom, the general formula (II) is represented by the following general formula (II-2).

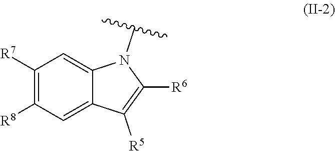

In the general formula (II), R⁵ represents a hydrogen atom, or a lower alkyl group.

As the lower alkyl group for R⁵, for example, a methyl group, an ethyl group, a propyl group, an isopropyl group, a butyl group, an isobutyl group, a sec-butyl group, a tert-butyl group, a pentyl group, an isopentyl group, an isoamyl group, a neopentyl group, a 1,1-dimethylpropyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,2,2-trimethylpropyl group, and a 1-ethyl-3-methylpropyl group, etc. are mentioned, and a methyl group is preferable.

R⁵ is preferably a hydrogen atom, or a methyl group.

In the general formula (II), R⁶ represents a hydrogen atom or a lower alkyl group, or R⁶ and R¹ may be together to form a group represented by the general formula: —(CH₂)ₚ— (wherein p is an integer of 1 or 2).

As the lower alkyl group for R⁶, for example, a methyl group, an ethyl group, a propyl group, an isopropyl group, a butyl group, an isobutyl group, a sec-butyl group, a tert-butyl group, a pentyl group, an isopentyl group, an isoamyl group, a neopentyl group, a 1,1-dimethylpropyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,2,2-trimethylpropyl group, a 1-ethyl-3-methylpropyl group, etc. are mentioned, and a methyl group is preferable.

The expression “R⁶ and R¹ may be together to form a group represented by the general formula: —(CH₂)ₚ—” means that a ring is provided by the group represented by the general formula: —(CH₂)ₚ— formed of R⁶ and R¹. When the “R⁶ and R¹ together form a group represented by the general formula: —(CH₂)ₚ—”, the general formula (I) is represented by, for example, the following general formula (I-9).

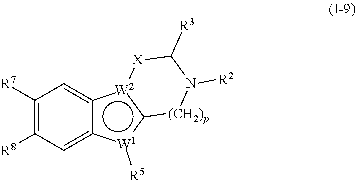

The general formula (I-9) corresponds to the general formula (I) (in the formula, A is a group represented by the general formula (II), R⁶ and R¹ are together to form a group represented by the general formula: —(CH₂)ₚ—, and further, p is an integer of 1 or 2, and preferably 2).

In the general formula (II), R⁷ represents a hydrogen atom, a halogen atom, a hydroxyl group, a lower alkoxy group, a carbamoyl group, or a group represented by the general formula: —Y—(CH₂)_{q}—R⁹.

As the halogen atom for R⁷, for example, a fluorine atom, a chlorine atom, a bromine atom, and an iodine atom are mentioned, and a fluorine atom, and a chlorine atom are preferable.

As the lower alkoxy group for R⁷, for example, a methoxy group, an ethoxy group, a propoxy group, an isopropoxy group, a butoxy group, a sec-butoxy group, a tert-butoxy group, a pentyloxy group, an isopentyloxy group, a hexyloxy group, an isohexyloxy group, etc. are mentioned, and a methoxy group is preferable.

Y represents a single bond, an oxygen atom, a sulfur atom, or a group represented by the general formula: —N(R^{N})— (wherein R^{N} represents a hydrogen atom, or a lower alkyl group).

q is an integer of 1 or 2, and preferably 1.

R⁹ represents a phenyl group, a thienyl group, or a furyl group.

R⁷ is preferably a hydrogen atom, a fluorine atom, a chlorine atom, a bromine atom, an iodine atom, a hydroxyl group, a methoxy group, an ethoxy group, a propoxy group, an isopropoxy group, a butoxy group, a sec-butoxy group, a tert-butoxy group, a pentyloxy group, an isopentyloxy group, a hexyloxy group, an isohexyloxy group, a carbamoyl group, a benzyl group, a thiophen-2-ylmethyl group, a thiophen-3-ylmethyl group, a furan-2-ylmethyl group, a furan-3-ylmethyl group, a benzyloxy group, a thiophen-2-ylmethoxy group, a thiophen-3-ylmethoxy group, a furan-2-ylmethoxy group, or a furan-3-ylmethoxy group, and more preferably is a hydrogen atom, a fluorine atom, a chlorine atom, a hydroxyl group, a methoxy group, a carbamoyl group, or a thiophen-2-ylmethoxy group.

In the general formula (II), R⁸ represents a hydrogen atom or a halogen atom.

As the halogen atom for R⁸, for example, a fluorine atom, a chlorine atom, a bromine atom, and an iodine atom are mentioned, and a fluorine atom is preferable.

R⁸ is preferably a hydrogen atom, or a fluorine atom.

In the general formula (III), R¹⁰ represents a hydrogen atom or a lower alkoxy group, or R¹⁰ and R¹ or R³ may be together to form a group represented by the general formula: —(CH₂)ᵣ— (wherein r is an integer of 1 or 2).

As the lower alkoxy group for R¹⁰, for example, a methoxy group, an ethoxy group, a propoxy group, an isopropoxy group, a butoxy group, a sec-butoxy group, a tert-butoxy group, a pentyloxy group, an isopentyloxy group, a hexyloxy group, and an isohexyloxy group, etc. are mentioned, and a methoxy group is preferable.

R¹⁰ is preferably a hydrogen atom, or a methoxy group.

The expression “R¹⁰ and R¹ may be together to form a group represented by the general formula: —(CH₂)ᵣ—” means that a ring is provided by the group represented by the general formula: —(CH₂)ᵣ— formed of R¹⁰ and R¹. When the “R¹⁰ and R¹ together form a group represented by the general formula: —(CH₂)ᵣ—”, the general formula (I) is represented by, for example, the following general formula (I-10).

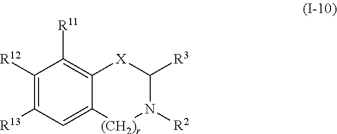

The general formula (I-10) corresponds to the general formula (I) (in the formula, A is a group represented by the general formula (III), and R¹⁰ and R¹ are together to form a group represented by the general formula —(CH₂)ᵣ—, and further, r is an integer of 1 or 2, and preferably 2).

The expression “R¹⁰ and R³ may be together to form a group represented by the general formula: —(CH₂)ᵣ” means that a ring is provided by the group represented by the general formula: —(CH₂)ᵣ— formed of R¹⁰ and R³. When the “R¹⁰ and R³ together to form a group represented by the general formula: —(CH₂)ᵣ—”, the general formula (I) is represented by, for example, the following general formula (I-11).

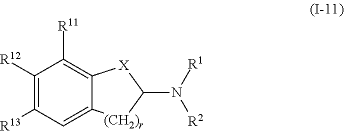

The general formula (I-11) corresponds to the general formula (I) (in the formula, A is a group represented by the general formula (III), and R¹⁰ and R³ are together to form a group represented by the general formula: —(CH₂)ᵣ—, and further, r is an integer of 1 or 2, and preferably 2).

In the general formula (III), R¹¹ represents a hydrogen atom or a hydroxyl group, or R¹¹ and R¹² may be together to form a propylene group.

The expression “R¹¹ and R¹² may be together to form a propylene group” means that a ring is provided by the propylene group formed of R¹¹ and R¹². As the specific example, the following general formula (III-1) can be mentioned.

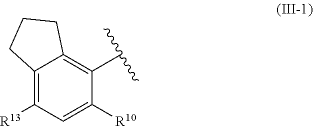

The general formula (III-1) corresponds to the general formula (III) (in the formula, R¹¹ and R¹² are together to form a propylene group).

In the general formula (III), R¹² represents a hydrogen atom, a halogen atom, a hydroxyl group, a lower alkoxy group, or a halo-lower alkyl group.

As the halogen atom for R¹², for example, a fluorine atom, a chlorine atom, a bromine atom, and an iodine atom are mentioned, and a chlorine atom is preferable.

As the lower alkoxy group for R¹², for example, a methoxy group, an ethoxy group, a propoxy group, an isopropoxy group, a butoxy group, a sec-butoxy group, a tert-butoxy group, a pentyloxy group, an isopentyloxy group, a hexyloxy group, an isohexyloxy group, etc. are mentioned, and a methoxy group is preferable.

As the halo-lower alkyl group for R¹², for example, a fluoromethyl group, a difluoromethyl group, a trifluoromethyl group, a 2-fluoroethyl group, a 1,2-difluoroethyl group, a 2,2,2-trifluoroethyl group, a pentafluoroethyl group, a chloromethyl group, a 2-chloroethyl group, a 1,2-dichloroethyl group, a 2,2,2-trichloroethyl group, a bromomethyl group, an iodomethyl group, etc. are mentioned, and a trifluoromethyl group is preferable.

R¹² is preferably a hydrogen atom, a chlorine atom, a hydroxyl group, a methoxy group, or a trifluoromethyl group.

In the general formula (III), R¹³ represents a hydrogen atom, a halogen atom, or a cyano group.

As the halogen atom for R¹³, for example, a fluorine atom, a chlorine atom, a bromine atom, and an iodine atom are mentioned, and a chlorine atom, a bromine atom, and an iodine atom are preferable.

R¹³ is preferably a hydrogen atom, a chlorine atom, a bromine atom, an iodine atom, or a cyano group.

Preferable examples of the compound represented by the general formula (I) include <1> to <18> in the following Table 1. Note that compounds of <19> to <21> each are not the compound represented by the general formula (I), but are examples of the compound having 5-HT_{2B} receptor agonist activity.

TABLE 1

| | Compound name | Another name |
|---|---|---|
| 1 | 3-(2-aminoethyl)-1H-indol-5-ol | 5-HT or serotonin |
| 2 | 3-(2-aminopropy1)-1H-indol-5-ol | α-methyl-5-HT |
| 3 | 2-(1H-indol-3-yl)ethan-1-amine | tryptamine |
| 4 | 3-(2-aminoethyl)-1H-indol-5-carboxamide | 5-CT |
| 5 | 3-(2-aminoethyl)-2-methyl-1H-indol-5-ol | 2-Me-5-HT |
| 6 | 1-(5-(thiophen-2-ylmethoxy)-1H-indol-3- | BW723C86 |
| | yl)propan-2-amine | |
| 7 | 5-methoxy-3-(1,2,3,6-tetrahydropyridin- | RU24969 |
| | 4-yl)-1H-indole | |
| 8 | (S)-1-(6-chloro-5-fluoro-1H-indol-1-yl) | Ro60-0175 |
| | propan-2-amine | |
| 9 | (S)-1-(5,6-difluoro-1H-indol-1-yl) | (S)-2-(5,6- |
| | propan-2-amine | difluoroindol-1-yl)- |
| | | 1-methylethylamine |
| 10 | 6-methyl-1,2,3,4,5,6-hexahydroazepino | PNU22394 |
| | [4,5-b]indole | |
| 11 | 1-(3-chlorophenyl)piperazine | MCPP |
| 12 | 1-(3-(trifluoromethyl)phenyl)piperazine | TFMPP |
| 13 | 1-(4-iodo-2,5-dimethoxyphenyl) | DOI |
| | propan-2-amine | |
| 14 | 1-(4-bromo-2,5-dimethoxyphenyl) | DOB |
| | propan-2-amine | |
| 15 | (R)-8-chloro-3-methyl-5-phenyl-2,3,4,5- | SCH23390 |
| | tetrahydro-1H-benzo[d]azepin-7-ol | |
| 16 | (R)-7-(dipropylamino)-5,6,7,8- | (R)-8-OH DPAT |
| | tetrahydronaphthalen-1-ol | |
| 17 | (S)-1-(3-(trifluoromethyl)phenyl) | nor-dexfenfluramine |
| | propan-2-amine | |
| 18 | (S)-3-((5-methoxy-2,3-dihydro-1H- | ORG-37684 |
| | inden-4-yl)oxy)pyrrolidine | |
| 19 | 2-chloro-6-(piperazin-1-yl)pyrazine | MK-212 |
| 20 | 1-(6-chloro-5-(trifluoromethyl) | ORG-12962 |
| | pyridin-2-yl)piperazine | |
| 21 | 2-(piperazin-1-yl)quinoline | Quipazine |

Chemical structures of the compounds <1> to <21> are shown in the following Table 2.

| | |
|---|---|
| 1 | 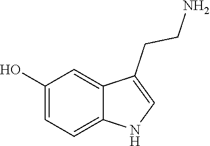 [SMILES: NCCc1c[nH]c2ccc(O)cc12] |
| 2 | 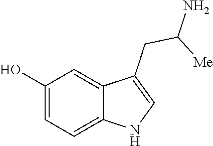 [SMILES: CC(N)Cc1c[nH]c2ccc(O)cc12] |
| 3 | 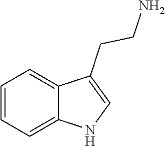 [SMILES: NCCc1c[nH]c2ccccc12] |
| 4 | 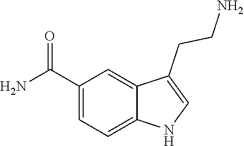 [SMILES: NCCc1c[nH]c2ccc(C(N)=O)cc12] |
| 5 | 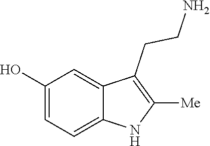 [SMILES: Cc1[nH]c2ccc(O)cc2c1CCN] |
| 6 | 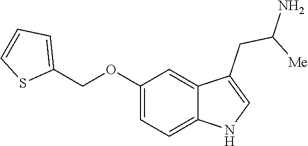 [SMILES: CC(N)Cc1c[nH]c2ccc(OCc3cccs3)cc12] |
| 7 | 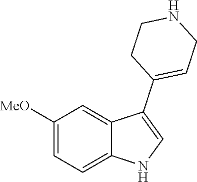 [SMILES: COc1ccc2[nH]cc(C3=CCNCC3)c2c1] |
| 8 | 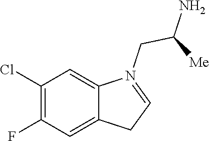 |
| 9 | 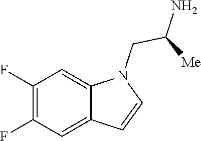 [SMILES: C[C@H](N)Cn1ccc2cc(F)c(F)cc21] |
| 10 | 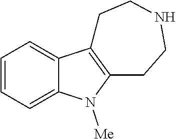 [SMILES: Cn1c2c(c3ccccc31)CCNCC2] |
| 11 | 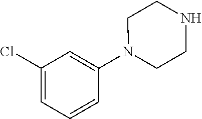 [SMILES: Clc1cccc(N2CCNCC2)c1] |
| 12 | 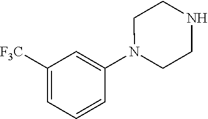 [SMILES: FC(F)(F)c1cccc(N2CCNCC2)c1] |
| 13 | 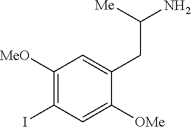 [SMILES: COc1cc(CC(C)N)c(OC)cc1I] |
| 14 | 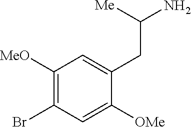 [SMILES: COc1cc(CC(C)N)c(OC)cc1Br] |
| 15 | 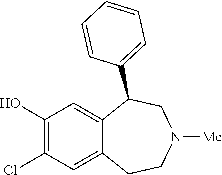 [SMILES: CN1CCc2cc(Cl)c(O)cc2[C@@H](c2ccccc2)C1] |
| 16 | 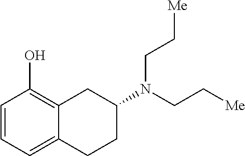 [SMILES: CCCN(CCC)[C@@H]1CCc2cccc(O)c2C1] |
| 17 | 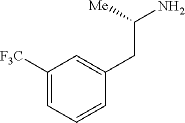 [SMILES: C[C@H](N)Cc1cccc(C(F)(F)F)c1] |
| 18 | 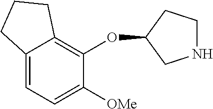 [SMILES: COc1ccc2c(c1O[C@H]1CCNC1)CCC2] |
| 19 | 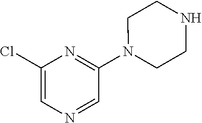 [SMILES: Clc1cncc(N2CCNCC2)n1] |
| 20 | 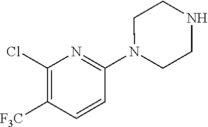 [SMILES: FC(F)(F)c1ccc(N2CCNCC2)nc1Cl] |
| 21 | 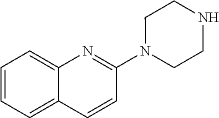 [SMILES: c1ccc2nc(N3CCNCC3)ccc2c1] |

Further, the relationships between the compounds <1> to <18> and the general formula (I) are shown in the following Table 3.

| | A | W¹ | W² | R⁵ | R⁶ | R⁷ | R⁸ | X | R³ | R¹ | R² |
|---|---|---|---|---|---|---|---|---|---|---|---|
| 1 | general | N | C | H | H | OH | H | —CH₂— | H | H | H |
| | formula | | | | | | | | | | |
| | (II) | | | | | | | | | | |
| 2 | general | N | C | H | H | OH | H | —CH₂— | CH₃ | H | H |
| | formula | | | | | | | | | | |
| | (II) | | | | | | | | | | |
| 3 | general | N | C | H | H | H | H | —CH₂— | H | H | H |
| | formula | | | | | | | | | | |
| | (II) | | | | | | | | | | |
| 4 | general | N | C | H | H | —CONH₂ | H | —CH₂— | H | H | H |
| | formula | | | | | | | | | | |
| | (II) | | | | | | | | | | |
| 5 | general | N | C | H | CH₃ | OH | H | —CH₂— | H | H | H |
| | formula | | | | | | | | | | |
| | (II) | | | | | | | | | | |
| 6 | general formula (II) | N | C | H | H | 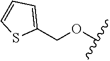 | H | —CH₂— | CH₃ | H | H |
| 7 | general | N | C | H | H | OMe | H | —C(R⁴)═CH— | H | R¹ and R⁴: | H |
| | formula | | | | | | | | | —CH₂CH₂— | |
| | (II) | | | | | | | | | | |
| 8 | general | C | N | H | H | Chlorine atom | Fluorine | —CH₂— | CH₃ | H | H |
| | formula | | | | | | atom | | | | |
| | (II) | | | | | | | | | | |
| 9 | general | C | N | H | H | Fluorine atom | Fluorine | —CH₂— | CH₃ | H | H |
| | formula | | | | | | atom | | | | |
| | (II) | | | | | | | | | | |
| 10 | general | N | C | CH₃ | R⁶ and R¹: | H | H | —CH₂— | H | R⁶ and R¹: | H |
| | formula | | | | —CH₂CH₂— | | | | | —CH₂CH₂— | |
| | (II) | | | | | | | | | | |

| | A | R¹⁰ | R¹¹ | R¹² | R¹³ | X | R³ | R¹ | R² |
|---|---|---|---|---|---|---|---|---|---|
| 11 | general | H | H | Chlorine | H | —N(R⁴)—CH₂— | H | R¹ and R⁴: | H |
| | formula | | | atom | | | | —CH₂CH₂— | |
| | (III) | | | | | | | | |
| 12 | general | H | H | CF₃ | H | —N(R⁴)—CH₂— | H | R¹ and R⁴: | H |
| | formula | | | | | | | —CH₂CH₂— | |
| | (III) | | | | | | | | |
| 13 | general | OMe | H | OMe | Iodine | —CH₂— | CH₃ | H | H |
| | formula | | | | atom | | | | |
| | (III) | | | | | | | | |
| 14 | general | OMe | H | OMe | Bromine | —CH₂— | CH₃ | H | H |
| | formula | | | | atom | | | | |
| | (III) | | | | | | | | |
| 15 | general | R¹⁰ and R¹: | H | OH | Chlorine | —CH(Ph)— | H | R¹⁰ and R¹: | CH₃ |
| | formula | —CH₂CH₂— | | | atom | | | —CH₂CH₂— | |
| | (III) | | | | | | | | |
| 16 | general | R¹⁰ and R³: | OH | H | H | —CH₂— | R¹⁰ and R³: | Propyl group | Propyl |
| | formula | —CH₂CH₂— | | | | | —CH₂CH₂— | | group |
| | (III) | | | | | | | | |
| 17 | general | H | H | CF₃ | H | —CH₂— | CH₃ | H | H |
| | formula | | | | | | | | |
| | (III) | | | | | | | | |
| 18 | general | OMe | R¹¹ and R¹²: | R¹¹ and R¹²: | H | —O—CH(R⁴)— | H | R¹ and R⁴: | H |
| | formula | | —(CH₂)₃— | —(CH₂)₃— | | | | —CH₂CH₂— | |
| | (III) | | | | | | | | |

As the pharmaceutically acceptable salt of the 5-HT_{2B} receptor agonist (hereinafter, also referred to as the “salt thereof”), for example, a hydrochloride, a maleate, a fumarate, and an oxalate, etc. are mentioned.

The pharmaceutically acceptable salt of the 5-HT_{2B} receptor agonist includes a solvate with a pharmaceutically acceptable solvent such as water or ethanol.

The 5-HT_{2B} receptor agonist and a salt thereof are known substances, and are easily available on the market, or easily synthesizable by a combination of known synthesis reactions.

<Muscle Regeneration Promoter>

The term “muscle regeneration promotion” used herein means that the regeneration of the damaged muscle tissue caused by muscle damage, myogenic disease, or the like is promoted.

As the muscle damage, for example, muscle strain (caused by external force (for example, bruise or the like)), pulled muscle (caused by internal force such as sudden contraction of muscle), and cervical sprain (so-called whiplash injury), are mentioned.

As the myogenic disease, for example, muscular dystrophy, and distal myopathy, etc. are mentioned.

In this regard, muscle damage and myogenic disease are common to each other in that muscle regeneration compensating for necrosis of muscle fibers (muscle damage) occurs.

The present invention is not bound by any specific theory, but since the 5-HT_{2B} receptor was expressed in muscle satellite cells that play a central role in muscle regeneration and differentiation and further the increase in the expression of 5-HT_{2B} receptor was observed during the muscle regeneration in a muscle-regeneration model animal (results of studies by the present inventors), it is considered that the muscle regeneration is promoted by the action of a 5-HT_{2B} receptor agonist on the receptor.

The concentration of the 5-HT_{2B} receptor agonist or a salt thereof in a muscle regeneration promoter can be appropriately set depending on the degree of muscle damage and the like.

The muscle regeneration promoter can be applied to an animal having muscle without any limitation. The application target is preferably a mammal (a human, or a non-human mammal (for example, a horse or a cow)), and more preferably a human. Further, there are no restrictions on the sex and age of the application target.

<Pharmaceutical Formulation>

The muscle regeneration promoter can be provided as a pharmaceutical formulation. The pharmaceutical formulation includes an oral formulation and a parenteral formulation. As the oral formulation, for example, a tablet, a capsule, powder, or granules can be mentioned. As the parenteral formulation, for example, a sterilized pharmaceutical formulation in a liquid state such as a solution or a suspension, specifically, an injection or an infusion can be mentioned. The pharmaceutical formulation is preferably an oral formulation, but in a case of the parenteral formulation, an intramuscular injection is preferred.

The pharmaceutical formulation may contain a pharmaceutically acceptable carrier or diluent together with an active ingredient. The formulation can be conducted by using a common formulation technique.

As the “pharmaceutically acceptable carrier or diluent”, for example, an excipient (for example, fat, beeswax, polyol of semi-solid or liquid, or natural or hardened oil); water (for example, distilled water, particularly, distilled water for injection); physiological saline; alcohol (for example, ethanol); glycerol; a polyol; an aqueous solution of glucose; mannitol; plant oil; and an additive agent (for example, a bulking agent, a disintegrant, a binding agent, a lubricant, a wetting agent, a stabilizer, an emulsifier, a dispersant, a preservative, a sweetener, a coloring agent, a seasoning or an aromatic substance, a thickener, a diluent, a buffer substance, a solvent, a solubilizer, a drug for achieving a storage effect, a salt for changing an osmotic pressure, a coating agent, or an antioxidant), etc. are mentioned.

The muscle regeneration promoter can be applied to various forms of pharmaceutical formulations. As the various forms, for example, an oral formulation (a tablet, a capsule, a powder, a granule, or a solution), a parenteral formulation (a sterilized solution or a suspension), a suppository, an ointment, etc. are mentioned.

The pharmaceutical formulation may be a solid formulation, or may also be a liquid formulation.

The solid formulation can be produced as it is in the form of a tablet, a capsule, a granule, or a powder, but can also be produced by using an appropriate carrier (additive). As the carrier (additive), for example, a saccharide (for example, lactose, or glucose); a starch (for example, maize, wheat, or rice); a fatty acid (for example, stearic acid); an inorganic salt (for example, magnesium aluminometasilicate, or anhydrous calcium phosphate); a synthetic polymer (for example, polyvinyl pyrrolidone, or polyalkylene glycol); a fatty acid salt (for example, calcium stearate, or magnesium stearate); an alcohol (for example, stearyl alcohol, or benzyl alcohol); a synthetic cellulose derivative (for example, methyl cellulose, carboxymethyl cellulose, ethyl cellulose, or hydroxypropyl methyl cellulose); and other usually-used additives (gelatin, talc, plant oil, and gum arabic), etc. are mentioned.

The solid preparation can contain, for example, 0.1 to 100% by mass, preferably 5 to 98% by mass of an active ingredient based on the total pharmaceutical formulation.

The liquid formulation can be produced in the form of a suspension, a syrup, an injection, an infusion (intravenous infusion), or the like by using an appropriate additive usually used in a liquid formulation (for example, water, an alcohol, or plant-derived oil such as soybean oil, peanut oil, or sesame oil).

As the appropriate solvent or diluent in a case of parenteral administration in the form of intramuscular injection, intravenous injection, or subcutaneous injection, for example, distilled water for injection, a lidocaine hydrochloride aqueous solution (for intramuscular injection), a saline solution, an aqueous solution of glucose, ethanol, polyethylene glycol, propylene glycol, a liquid for intravenous injection (for example, an aqueous solution of citric acid, sodium citrate, or the like), an electrolyte solution (for intravenous drip infusion or intravenous injection), and a mixture solution thereof, etc. are mentioned.

These injections may be prepared in the form of pre-dissolved active ingredient, and further may be prepared in the form that is dissolved at the time of use as a powder of the active ingredient as it is or a powder of the active ingredient added with an appropriate carrier (additive). The injection can contain, for example, 0.005 to 25% by mass of an active ingredient based on the total pharmaceutical formulation.

<Therapeutic Agent for Muscle Damage>

The 5-HT_{2B} receptor agonist and a salt thereof can treat muscle damage by promoting the regeneration of the damaged muscle tissue. Accordingly, the muscle regeneration promoter according to the present invention can be grasped also as a therapeutic agent for muscle damage.

The description about the active ingredient and formulation of the muscle regeneration promoter is applied to the therapeutic agent for muscle damage.

### EXAMPLES

Next, the effects of the present invention will be specifically described by way of Examples, however, the present invention is not limited to these Examples.

<Experimental Method>

1. Evaluation Compounds

The following 4 kinds of compounds were evaluated.

| Example | Compound name | Note |
|---|---|---|
| 1 | BW723C86 | Compound 6 in Table 1 |
| 2 | α-methyl-5-HT | Compound 2 in Table 1 |
| 3 | Ro 60-0175 | Compound 8 in Table 1 |
| 4 | PNU 22394 | Compound 10 in Table 1 |

2. Test Animals

Seven-week old C57BL/6 male mice (CLEA Japan, Inc.) were purchased, and used for experiment at the age of 8 weeks.

3. Muscle-Damaged Model Animal

A model animal to which muscle damage had been caused by administration of snake venom cardiotoxin (CTX) was used. The muscle-damaged model animal has been widely used in studies on the regeneration from muscle damage.

Under the anesthesia with isoflurane, 50 μL of 10 μM CTX was administered to the tibialis anterior muscle of the right hindlimb of the mouse. After 7 days of the administration of CTX, the tibialis anterior muscle was collected by dissection, and supplied to the preparation of a muscle tissue section.

4. Preparation of Muscle Tissue Section

Immediately after the collection of the tibialis anterior muscle, the tibialis anterior muscle was immersed in isopentane cooled with liquid nitrogen and was rapidly frozen. The frozen muscle tissue was cut into slices each having a thickness of 10 μm by using a cryostat (Leica Biosystems), and the slice was attached onto an antistripping coated slide glass (Matsunami Glass Ind., Ltd).

5. Immunofluorescent Staining of Muscle Tissue Section

A muscle tissue section was sufficiently air dried for 30 minutes under room temperature. After that, the muscle tissue section was fixed by immersing it in acetone cooled to −30° C. and treating at −30° C. for 20 minutes. The fixed section was air dried once and washed with PBS. Then the section was blocked by dropwisely adding a blocking reagent (Blocking One, NACALAI TESQUE, INC.) to the section and being subjected to the blocking treatment for 1 hour. Next, a primary antibody (Anti-Laminin-2 (α-2 Chain) antibody, Rat monoclonal (Sigma-Aldrich)) obtained by being diluted 500 times with the blocking reagent was added dropwise, and the reaction was conducted at overnight at 4° C. Since laminin to which a primary antibody binds is a protein expressed in all muscle cells, the primary antibody was used in this experiment in order to measure the area of individual muscle cells in a section. The muscle tissue section after the reaction with the primary antibody was washed with PBS, and then was reacted for 1 hour with a secondary antibody (CF 488A Goat Anti-Rat IgG (H+L) (Biotium)) obtained by being diluted 500 times with the blocking reagent. The secondary antibody that is an anti-rat antibody conjugated with a fluorescent dye binds to the primary antibody, and stains the laminin. The muscle tissue section after the reaction with the secondary antibody was washed with PBS, and sealed by using “VECTASHIELD Hard⋅Set with DAPI” (Vector), and then the fluorescence observation was performed with an inverted microscope FSX100 (OLYMPUS). The “VECTASHIELD Hard⋅Set with DAPI” was used for staining the central nucleus of muscle cells.

6. Evaluation for Muscle Regeneration

The muscle regeneration was evaluated on the basis of the image data taken from the fluorescence observation. In this experiment, muscle cells each having a central nucleus (single muscle fibers having central nuclei) were used as an indicator for regenerated muscle. After the image data was taken into image analysis software ImageJ (NIH), a muscle cell having a central nucleus was extracted. The cross-sectional area of the extracted individual cells was measured on the basis of the cell membrane stained with laminin. For the area measurement, “Analyze Particles” that is an add-in analysis program on ImageJ was used. The measurement results were shown as a distribution chart (histogram) of the area and number of regenerated single-muscle fibers, and as an average value of the cross-sectional areas of all the regenerated single muscle fibers (mean muscle fiber area).

### Example 1

### Muscle Regeneration Promotion Effect of BW723C86>

BW723C86 is known to be a selective 5-HT_{2B} receptor agonist (Br. J. Pharmacol., Vol. 128, No. 1, pp. 13-20, September 1999).

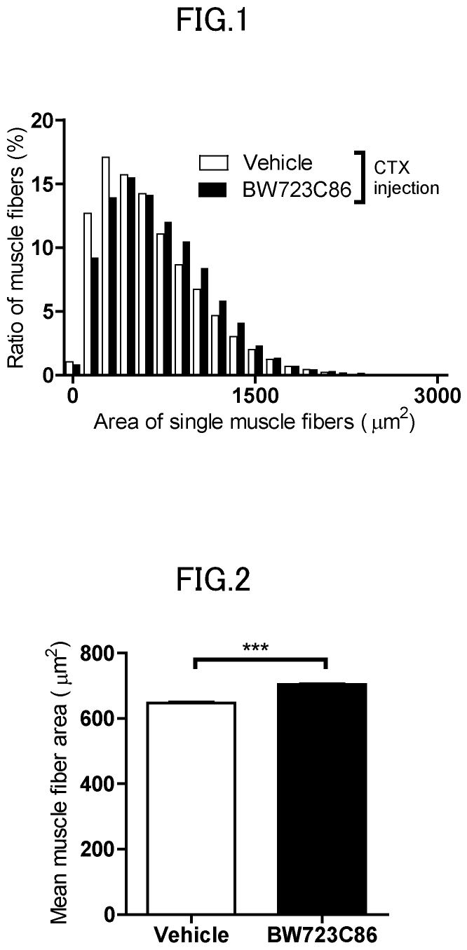

BW723C86 hydrochloride (Tocris) dissolved in a PBS with 5% dimethyl sulfoxide at a concentration of 5 mM was injected intramuscularly in a volume of 10 μL into the tibialis anterior muscle of both legs of a mouse once a day from the day before CTX administration to the day before dissection (6 day after CTX administration). In a control group (Vehicle), a PBS with 5% dimethyl sulfoxide solution was injected intramuscularly into the tibialis anterior muscle of both legs of a mouse. The number and area of regenerated single muscle fibers in the collected tibialis anterior muscle were measured by the above method. The results are shown in FIGS. 1 and 2.

When the muscle regeneration was evaluated on the basis of the histogram of the single-muscle fiber area, the histogram of the BW723C86 administration group was shifted to the right side (in a direction in which the area becomes larger) as compared with the histogram of the Vehicle administration group (FIG. 1).

When the muscle regeneration was evaluated on the basis of the mean muscle fiber area, a significant increase in the mean muscle fiber area (9.5%) was observed in the BW723C86 administration group as compared with the Vehicle administration group (FIG. 2, *** P<0.0001).

These results indicate that BW723C86 promoted the increase of the area of muscle regenerated from the damage due to CTX administration, that is, muscle regeneration.

### Example 2

### Muscle Regeneration Promotion Effect of α-methyl-5-HT>

α-methyl-5-HT is known to be a 5-HT_{2B} receptor agonist (Br. J. Pharmacol., Vol. 128, No. 1, pp. 13-20, September 1999).

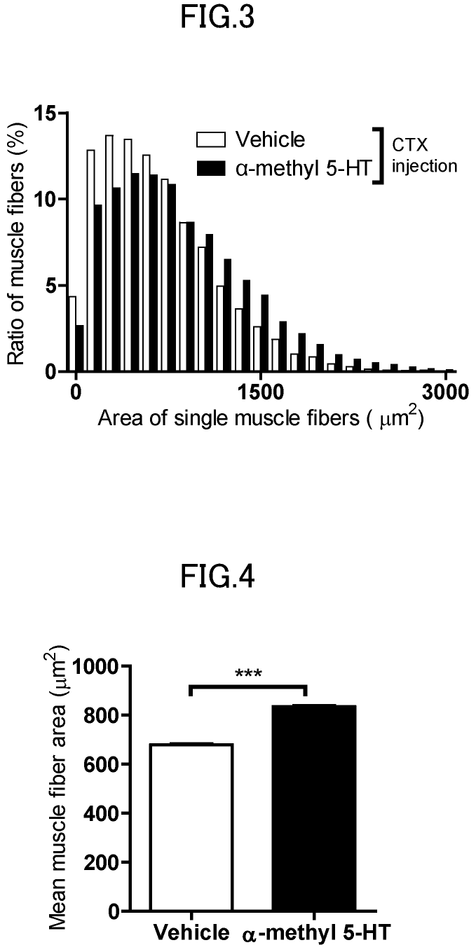

α-methyl-5-HT malate (Sigma-Aldrich) dissolved in a PBS with 5% dimethyl sulfoxide solution at a concentration of 5 mM was injected intramuscularly in a volume of 10 μL into the tibialis anterior muscle of both legs of a mouse once a day from the day before CTX administration to the day before dissection (6 day after CTX administration). In a control group (Vehicle), a PBS with 5% dimethyl sulfoxide solution was injected intramuscularly into the tibialis anterior muscle of both legs of a mouse. The number and area of regenerated single muscle fibers in the collected tibialis anterior muscle were measured by the above method. The results are shown in FIGS. 3 and 4.

When the muscle regeneration was evaluated on the basis of the histogram of the single-muscle fiber area, the histogram of the α-methyl-5-HT administration group was shifted to the right side (in a direction in which the area becomes larger) as compared with the histogram of the Vehicle administration group (FIG. 3).

When the muscle regeneration was evaluated on the basis of the mean muscle fiber area, a significant increase in the mean muscle fiber area (23%) was observed in the α-methyl-5-HT administration group as compared with the Vehicle administration group (FIG. 4, ***P<0.0001).

These results indicate that α-methyl-5-HT promoted the increase of the area of muscle regenerated from the damage due to CTX administration, that is, muscle regeneration.

### Example 3

### Muscle Regeneration Promotion Effect of Ro 60-0175>

Ro 60-0175 is known to be a 5-HT_{2B} receptor agonist (Br. J. Pharmacol., Vol. 128, No. 1, pp. 13-20, September 1999).

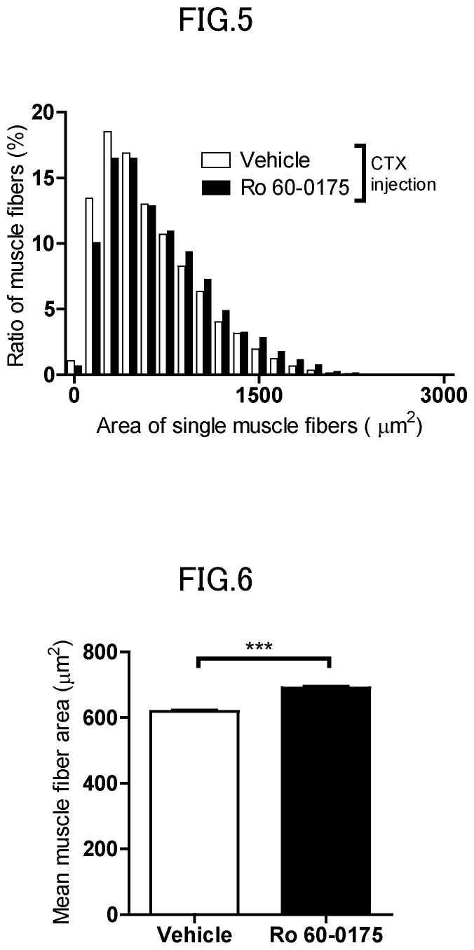

Ro 60-0175 fumarate (Tocris) dissolved in a PBS with 5% dimethyl sulfoxide solution at a concentration of 5 mM was injected intramuscularly in a volume of 10 μL into the tibialis anterior muscle of both legs of a mouse once a day from the day before CTX administration to the day before dissection (6 day after CTX administration). In a control group (Vehicle), a PBS with 5% dimethyl sulfoxide solution was injected intramuscularly into the tibialis anterior muscle of both legs of a mouse. The number and area of regenerated single muscle fibers in the collected tibialis anterior muscle were measured by the above method. The results are shown in FIGS. 5 and 6.

When the muscle regeneration was evaluated on the basis of the histogram of the single-muscle fiber area, the histogram of the Ro 60-0175 administration group was shifted to the right side (in a direction in which the area becomes larger) as compared with the histogram of the Vehicle administration group (FIG. 5).

When the muscle regeneration was evaluated on the basis of the mean muscle fiber area, a significant increase in the mean muscle fiber area (12%) was observed in the Ro 60-0175 administration group as compared with the Vehicle administration group (FIG. 6, ***P<0.0001).

These results indicate that Ro 60-0175 promoted the increase of the area of muscle regenerated from the damage due to CTX administration, that is, muscle regeneration.

### Example 4

### Muscle Regeneration Promotion Effect of PNU 22394>

PNU 22394 is known to be a 5-HT_{2B} receptor agonist (J. Pharmacol. Exp. Ther., Vol.347, No.3, pp.645-59, December 2013).

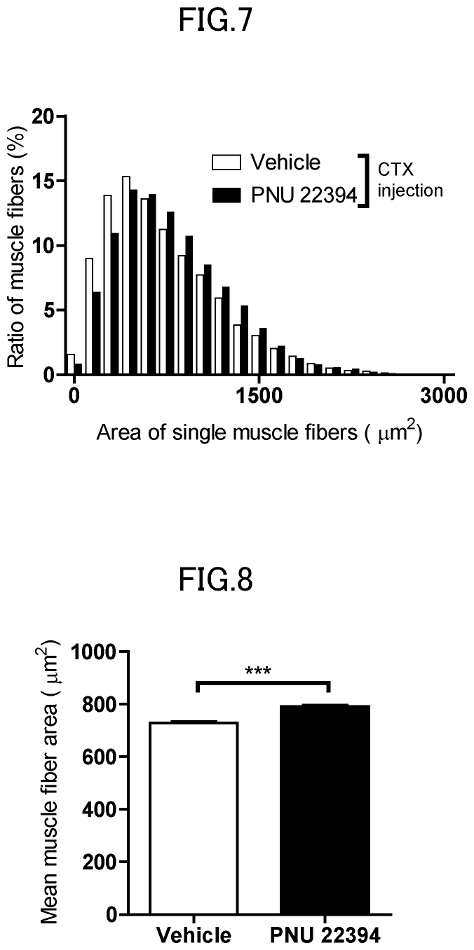

PNU 22394 hydrochloride (Tocris) dissolved in PBS at a concentration of 10 mM was injected intramuscularly in a volume of 10 μL into the tibialis anterior muscle of both legs of a mouse once a day from the day before CTX administration to the day before dissection (6 day after CTX administration). In a control group (Vehicle), PBS was injected intramuscularly into the tibialis anterior muscle of both legs of a mouse. The number and area of regenerated single muscle fibers in the collected tibialis anterior muscle were measured by the above method. The results are shown in FIGS. 7 and 8.

When the muscle regeneration was evaluated on the basis of the histogram of the single-muscle fiber area, the histogram of the PNU 22394 administration group was shifted to the right side (in a direction in which the area becomes larger) as compared with the histogram of the Vehicle administration group (FIG. 7).

When the muscle regeneration was evaluated on the basis of the mean muscle fiber area, a significant increase in the mean muscle fiber area (8.5%) was observed in the PNU 22394 administration group as compared with the Vehicle administration group (FIG. 8, ***P<0.0001).

These results indicate that PNU 22394 promoted the increase of the area of muscle regenerated from the damage due to CTX administration, that is, muscle regeneration.

### INDUSTRIAL APPLICABILITY

By using the muscle regeneration promoter according to the present invention, the return to daily life and sports activities of patients with muscle damage can be accelerated. Therefore, the present invention can be used in the treatment of muscle damage.

1. A method for promoting muscle regeneration in an animal, comprising a step of administering a compound having 5-HT_{2B} receptor agonist activity or a pharmaceutically acceptable salt thereof to the aminal.

2. The method according to claim 1, wherein
the compound having 5-HT_{2B} receptor agonist activity is a compound represented by the general formula (I): 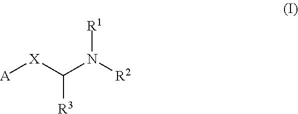
[wherein
R¹ represents a hydrogen atom, a lower alkyl group, or a group represented by the general formula: —CH₂Ar (wherein Ar represents a phenyl group that may be substituted with a hydroxyl group);
R² represents a hydrogen atom, or a lower alkyl group;
R³ represents a hydrogen atom, or a lower alkyl group;
X represents a methylene group, a group represented by the general formula: —C(R⁴)═CH—, a group represented by the general formula: —N(R⁴)—CH₂—, a group represented by the general formula: —O—CH(R⁴)—, or a group represented by the general formula: —CH(R⁴)—;
R⁴ represents a hydrogen atom or a phenyl group, or R⁴ and R¹ may be together to form a group represented by the general formula: —(CH₂)ₙ— (wherein n is an integer of 1 or 2); and
A is a group represented by the general formula (II): 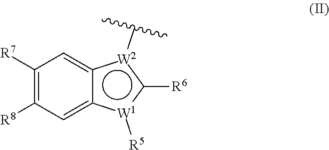
(wherein
W¹ and W² each represent independently a nitrogen atom, or a carbon atom (wherein when one of W¹ and W² represents a nitrogen atom, the remaining represents a carbon atom);
R⁵ represents a hydrogen atom, or a lower alkyl group;
R⁶ represents a hydrogen atom or a lower alkyl group, or R⁶ and R¹ may be together to form a group represented by the general formula: —(CH₂)ₚ— (wherein p is an integer of 1 or 2);
R⁷ represents a hydrogen atom, a halogen atom, a hydroxyl group, a lower alkoxy group, a carbamoyl group, or a group represented by the general formula: —Y—(CH₂)_{q}—R⁹ (wherein Y represents a single bond, an oxygen atom, a sulfur atom, or a group represented by the general formula: —N(R^{N})— (wherein R^{N} represents a hydrogen atom or a lower alkyl group), and q is an integer of 1 or 2);
R⁸ represents a hydrogen atom, or a halogen atom; and
R⁹ represents a phenyl group, a thienyl group, or a furyl group); or
a group represented by the general formula (III): 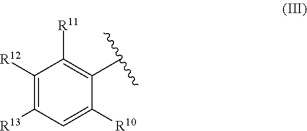
(wherein
R¹⁰ represents a hydrogen atom or a lower alkoxy group, or R¹⁰ and R¹ or R³ may be together to form a group represented by the general formula: —(CH₂)ᵣ— (wherein r is an integer of 1 or 2);
R¹¹ represents a hydrogen atom or a hydroxyl group, or R¹¹ and R¹² may together to form a propylene group;
R¹² represents a hydrogen atom, a halogen atom, a hydroxyl group, a lower alkoxy group, or a halo-lower alkyl group; and
R¹³ represents a hydrogen atom, a halogen atom, or a cyano group)].

3. The method according to claim 2, wherein
A represents a group represented by the general formula (II).

4. The method according to claim 1, wherein
the compound having 5-HT_{2B} receptor agonist activity is selected from the group consisting of the following compounds <1> to <21>:
<1> 3-(2-aminoethyl)-1H-indol-5-ol,
<2> 3-(2-aminopropyl)-1H-indol-5-ol,
<3> 2-(1H-indol-3-yl)ethan-1-amine,
<4> 3-(2-aminoethyl)-1H-indol-5-carboxamide,
<5> 3-(2-aminoethyl)-2-methyl-1H-indol-5-ol,
<6> 1-(5-(thiophen-2-ylmethoxy)-1H-indol-3-yl)propan-2-amine,
<7> 5-methoxy-3-(1,2,3,6-tetrahydropyridin-4-yl)-1H-indole,
<8> (S)-1-(6-chloro-5-fluoro-1H-indol-1-yl)propan-2-amine,
<9> (S)-1-(5,6-difluoro-1H-indol-1-yl)propan-2-amine,
<10> 6-methyl-1,2,3,4,5,6-hexahydroazepino[4,5-b]indole,
<11> 1-(3-chlorophenyl)piperazine,
<12> 1-(3-(trifluoromethyl)phenyl)piperazine,
<13> 1-(4-iodo-2,5-dimethoxyphenyl)propan-2-amine,
<14> 1-(4-bromo-2,5-dimethoxyphenyl)propan-2-amine,
<15> (R)-8-chloro-3-methyl-5-phenyl-2,3,4,5-tetrahydro-1H-benzo[d]azepin-7-ol,
<16> (R)-7-(dipropylamino)-5,6,7,8-tetrahydronaphthalen-1-ol,
<17> (S)-1-(3-(trifluoromethyl)phenyl)propan-2-amine,
<18> (S)-3-((5-methoxy-2,3-dihydro-1H-inden-4-yl)oxy)pyrrolidine,
<19> 2-chloro-6-(piperazin-1-yl)pyrazine,
<20> 1-(6-chloro-5-(trifluoromethyl)pyridin-2-yl)piperazine, and
<21> 2-(piperazin-1-yl)quinoline.

5. The method according to claim 1, wherein
the compound having 5-HT_{2B} receptor agonist activity is selected from the group consisting of the following compounds <a> to <d>:
<a> 3-(2-aminopropyl)-1H-indol-5-ol,
<b> 1-(5-(thiophen-2-ylmethoxy)-1H-indol-3-yl)propan-2-amine,
<c> (S)-1-(6-chloro-5-fluoro-1H-indol-1-yl)propan-2-amine, and
<d> 6-methyl-1,2,3,4,5,6-hexahydroazepino[4,5-b]indole.

6. The method according to claim 1, wherein muscle regeneration after muscle damage or in myogenic disease is promoted.

7. The method according to claim 6, wherein the muscle regeneration after muscle damage is promoted.

8. The method according to claim 7, wherein the muscle damage is muscle strain.

9. A method for treating muscle damage in an animal, comprising a step of administering a compound having 5-HT_{2B} receptor agonist activity, or a pharmaceutically acceptable salt thereof to the animal.

10. The method according to claim 9, wherein the compound having 5-HT_{2B} receptor agonist activity is a compound represented by the general formula (I): 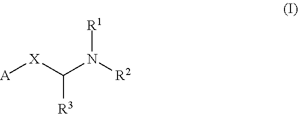
[wherein
R¹ represents a hydrogen atom, a lower alkyl group, or a group represented by the general formula: —CH₂Ar (wherein Ar represents a phenyl group that may be substituted with a hydroxyl group);
R² represents a hydrogen atom, or a lower alkyl group;
R³ represents a hydrogen atom, or a lower alkyl group;
X represents a methylene group, a group represented by the general formula: —C(R⁴)═CH—, a group represented by the general formula: —N(R⁴)—CH₂—, a group represented by the general formula: —O—CH(R⁴)—, or a group represented by the general formula: —CH(R⁴)—;
R⁴ represents a hydrogen atom or a phenyl group, or R⁴ and R¹ may be together to form a group represented by the general formula: —(CH₂)ₙ— (wherein n is an integer of 1 or 2); and
A is a group represented by the general formula (II): 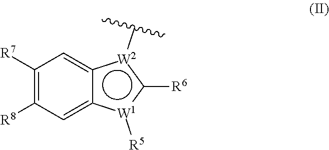
(wherein
W¹ and W² each represent independently a nitrogen atom, or a carbon atom (wherein when one of W¹ and W² represents a nitrogen atom, the remaining represents a carbon atom);
R⁵ represents a hydrogen atom, or a lower alkyl group;
R⁶ represents a hydrogen atom or a lower alkyl group, or R⁶ and R¹ may be together to form a group represented by the general formula: —(CH₂)ₚ— (wherein p is an integer of 1 or 2);
R⁷ represents a hydrogen atom, a halogen atom, a hydroxyl group, a lower alkoxy group, a carbamoyl group, or a group represented by the general formula: —Y—(CH₂)_{q}—R⁹ (wherein Y represents a single bond, an oxygen atom, a sulfur atom, or a group represented by the general formula: —N(R^{N})— (wherein R^{N} represents a hydrogen atom or a lower alkyl group), and q is an integer of 1 or 2);
R⁸ represents a hydrogen atom, or a halogen atom; and
R⁹ represents a phenyl group, a thienyl group, or a furyl group); or
a group represented by the general formula (III): 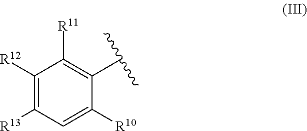
(wherein
R¹⁰ represents a hydrogen atom or a lower alkoxy group, or R¹⁰ and R¹ or R³ may be together to form a group represented by the general formula: —(CH₂)ᵣ— (wherein r is an integer of 1 or 2);
R¹¹ represents a hydrogen atom or a hydroxyl group, or R¹¹ and R¹² may be together to form a propylene group;
R¹² represents a hydrogen atom, a halogen atom, a hydroxyl group, a lower alkoxy group, or a halo-lower alkyl group; and
R¹³ represents a hydrogen atom, a halogen atom, or a cyano group)].

11. The method according to claim 9, wherein
the muscle damage is muscle strain.
